(19) United States
(12) Patent Application Publication
(10) Pub. No.: US 2009/0087911 A1
(43) Pub. Date: Apr. 2, 2009

(54) CODED OPTICAL EMISSION PARTICLES FOR SUBSURFACE USE

(75) Inventor: Rogerio T. Ramos, Hampshire (GB)

Correspondence Address: SCHLUMBERGER RESERVOIR COMPLETIONS, 14910 AIRLINE ROAD, ROSHARON, TX, 77583, US

(73) Assignee: SCHLUMBERGER TECHNOLOGY CORPORATION, Sugar Land, TX (US)

(21) Appl. No.: 11/863,598

(22) Filed: Sep. 28, 2007

(51) Int. Cl. G01N 33/24; B01J 19/00

(52) U.S. Cl. 436/27; 422/82.05; 422/72

(57) ABSTRACT

Tagging system and method including a plurality of particles, each particle having a miniature body and configured to provide a non-radioactive resolvable optical emission in a distinguishable pattern when selectively illuminated. The particles are set for selective release to a subsurface location. An apparatus having an elongated body configured for subsurface disposal and a chamber to house a plurality of particles therein.

### BACKGROUND

1. Technical Field

The present invention relates generally to the field of tracers or marker materials. More specifically, the invention relates to subsurface tagging and monitoring techniques.

2. Description of Related Art

Tracers have been used in the oil and gas industry for many years. One conventional technique has been to use radioactive substances as tracers, which has not always been possible due to safety and environmental considerations. U.S. Pat. No. 5,243,190 describes the use of radioactive particles for subsurface tracers. One of the uses of tracers has been to determine the “lag time” of the drilling fluid (“mud”) to travel from the surface down the borehole, through the drill bit and up to the surface again. A conventional technique for this purpose entails the injection of calcium carbide pellets, enclosed in a water-proof container, at the surface of the well being drilled for transit down the borehole by the mud stream. When passing through the drill bit, the container is smashed releasing the calcium carbide that reacts with water in the mud to form a gas, acetylene, which is detected at the surface with a gas analyzer. The lag time can therefore be determined from the time difference between the injection of the calcium carbide in the well and the detection of gas at the surface in the return mud.

Another conventional use of tracers relates to the injection of tracers in one well, followed by their detection in an adjacent well so as to make well-to-well correlations, enabling the characterization of the underground formation traversed by the two wells. Various chemicals have been used as tracers in subsurface applications. For example, U.S. Pat. No. 4,447,340 describes a method of tracing well drilling mud by determining the concentration of acetate tracer ion in the penetrated strata (by core analysis). The use as tracers of dichromate, chromate, nitrate, ammonium, cobalt, nickel, manganese, vanadium and lithium is also mentioned.

Some tracer techniques have also been proposed using spectroscopic techniques, including atomic absorption spectroscopy, X-ray fluorescence spectroscopy, or neutron activation analysis, to identify certain materials as tagging agents. U.S. Pat. No. 6,725,926 proposes the use of a proppant coated with phosphorescent, fluorescent, or photoluminescent pigments that glow in the dark upon exposure to certain lighting. Fluorescence spectrometry techniques entailing the illumination of fluids with a light source have also been proposed (See U.S. Pat. Nos. 7,084,392, 6,707,556, 6,564,866, 6,955,217, U.S. Patent Publication No. 20060054317).

Conventional tracer techniques have been limited by the variety of codes that can be used—normally only one. A need remains for improved tracer/tagging techniques, particularly in the areas of oil, gas, and water exploration and production.

### SUMMARY

One aspect of the invention provides a tagging system. The system includes a plurality of particles. Each particle having a miniature body and configured to provide a non-radioactive resolvable optical emission in a distinguishable pattern when selectively illuminated; and wherein one or more of the particles are set for selective release to a subsurface location.

Another aspect of the invention provides a tagging method. The method includes setting a plurality of particles, each particle having a miniature body and configured to provide a non-radioactive resolvable optical emission in a distinguishable pattern when selectively illuminated; and selectively releasing one or more of the particles for subsurface disposal.

Another aspect of the invention provides an apparatus. The apparatus includes an elongated body configured for subsurface disposal. The body having at least one chamber to house a plurality of particles therein, each particle having a miniature body and configured to provide a non-radioactive resolvable optical emission in a distinguishable pattern when selectively illuminated.

### BRIEF DESCRIPTION OF THE DRAWINGS

Other aspects and advantages of the invention will become apparent upon reading the following detailed description and upon reference to the drawings in which like elements have been given like numerals and wherein:

FIG. 1 is a schematic of particles revealing a coded pattern of fluorescence emission in response to illumination by a light source in accordance with aspects of the invention.

FIG. 2 is a schematic of a well drilling system including coded particle release units and a particle detection unit in accordance with aspects of the invention.

FIG. 3 is a schematic of a coded particle release unit in accordance with aspects of the invention.

FIG. 4 is a schematic of a downhole tool including coded particle release and detection units in accordance with aspects of the invention.

FIG. 5 is a schematic of another downhole tool including coded particle release units in accordance with aspects of the invention.

FIG. 6 is a schematic of a downhole tool including a coded particle release unit and implemented in a well-to-well application in accordance with aspects of the invention.

FIG. 7 is a flow chart of a tagging method in accordance with aspects of the invention.

### DETAILED DESCRIPTION

The present invention comprises the implementation of new coded or tagged particle technology. Aspects of the invention use small particles doped with different substances, such as rare earth elements, that can provide unique patterns of optical emission when excited with the appropriate wavelength radiation. The coded particle technology of the invention is described in M. J. Dejneka et al., Optically active glasses for biology, 3-D display, and telecommunications, Proceedings of the XX ICG International Congress on Glass, Kyoto, Sep. 27 to Oct. 1, 2004; M. J. Dejneka et al., Rare earth-doped glass microbarcodes, Proceedings of the National Academy of Sciences of the United States of America, PNAS, Jan. 21, 2003, vol. 100, no. 2, 389-393 [hereinafter “the Dejneka Papers”], both entirely incorporated herein by reference. Aspects of the invention disclosed herein are based on technology described in great detail by the Dejneka Papers.

Aspects of the invention entail micrometer-sized barcodes (microbarcodes) containing a pattern of different fluorescent materials that are easily identified by illumination with radiation of certain wavelengths. One aspect is implemented using a UV lamp and an optical microscope. Rare earth (RE) ions in a silicate glass matrix present an ideal material for the fabrication of encoded particles. For purposes of this disclosure, the term “particle” is understood to comprise an element or composition generally miniature in size and configured in any of various shapes and dimensions (e.g., a ball, bead, rod, ribbon, sphere, globule, droplet, tube). Similarly, the term “subsurface” is herein understood as, relating to, or situated in an area beneath a surface, especially the surface of the earth or of a body of water. For example, a subsurtace component is understood to comprise a buried, submerged, or partially buried/submerged component. RE-doped glasses were chosen because of their narrow emission bands, high quantum efficiencies, noninterference with common fluorescent labels, and inertness to most organic and aqueous solvents. These properties and the large number (greater than 1 million) of possible combinations of the microbarcodes make them attractive for use in subsurface encoding applications.

As described in the Dejneka Papers, REs are a spectroscopically rich species, which makes their use as optical codes in a spectral window distinct from fluorescent dyes. REs allow more resolvable bands to be packed into the same spectral bandwidth, which enables a larger number of distinct combinations for coding applications. They are also resistant to photobleaching. Multiple RE ions can be simultaneously excited in the UV spectrum and conveniently decoded by observing their emission in the visible, without interfering with other materials that have excitations in the visible. A silica-based glass matrix for the particles offers advantages, including compatibility with organic solvents, and low background fluorescence that provides lower limits of detection. Glass preforms can also be drawn down into extensions of very thin fiber or ribbons whose structure is an exact miniature of the parent preform, allowing large complex structures to be replicated down to the desired size.

Glass fabrication for aspects of the particles comprises mixing RE-doped alkaline earth aluminosilicate glass compositions for a particular color. Conventional optical fiber draw methods may be used to fabricate encoded fiber ribbons. As described in the Dejneka Papers, the optimized glasses are melted, cast, and annealed. The assembly is fused in a furnace and the preform is drawn into a ribbon fiber (20 μm thick, 100 μm wide). The ribbon fiber is scribed every 20 μm with laser pulses using a computer-controlled stage. The scribed ribbon fiber is then sonicated in water to break the ribbon along the scribes into individual barcodes.

Fabrication of RE-doped barcodes is not limited to the use of a silica-based matrix or fiber draw techniques, alternative approaches are possible using other materials and techniques. For example, other aspects of the invention may be implemented with one or more particles 10 comprising a matrix based on any other type of glass, crystal glass, crystal, a type of silicon oxide, germanium oxide, aluminum oxide, boron oxide, ceramic, or polymer. Yet other aspects may be implemented wherein the particle 10 matrix comprises a ferromagnetic material. With such an embodiment, a magnetic field may be used to collect or extract the particles 10 for analysis.

The barcoded particles can be decoded and imaged by using a spectral imager and a fluorescence microscope equipped with a mercury lamp. A dichroic filter may be used to select the excitation wavelength. A 420-nm long-pass filter has been used to observe RE fluorescence. It will be appreciated by those skilled in the art that various combinations of filters and imaging equipment may be used in aspects of the invention.

Candidate RE ions for the microbarcode particles of the invention preferably have nonoverlapping, bright visible luminescence for ease of detection, a common excitation source for simultaneous interrogation of observed barcode elements, and no overlap of excitation (and/or emission). Multiplexed excitation of the particles can be implemented using a UV radiation source. A usable UV light source is a mercury lamp (e.g., one that emits at 254 and 365 nm). As illustrated in the Dejneka Paper (Rare earth-doped glass microbarcodes), upon illumination by UV light, the fluorescence spectra of glasses doped with Ce³⁺, Tm³⁺, Tb³⁺, and Dy³⁰⁺ are well resolved and easily distinguished with the naked eye. The UV excitation respectively makes the Ce³⁺, Tm³⁺, Tb³⁺, and Dy⁺-doped glasses glow cyan, blue, green, and pale orange/yellow.

The coded particles of the invention can be configured with an extremely high number of barcodes by varying the scribe-length of the ribbon, the number of bands in a ribbon, and the concentration of the candidate elements. One aspect can be configured for coding involving a simple binary-type “yes/no” determination of color and sequence within a ribbon. The Dejneka Paper (Rare earth-doped glass microbarcodes) describes the use of five “combination colors”: binary combinations of Ce³⁺—Tb³⁺, Ce³⁺—Dy³⁺, Tm³⁺—Tb³⁺, Tm³⁺—Dy³⁺, and Tb³⁺—Dy³⁺. These doped glasses produce clearly resolvable fluorescence and negligible quenching. With these encoding options, fabrication of>10⁶ uniquely distinguishable barcodes by using RE-doped glass fibers is theoretically achievable.

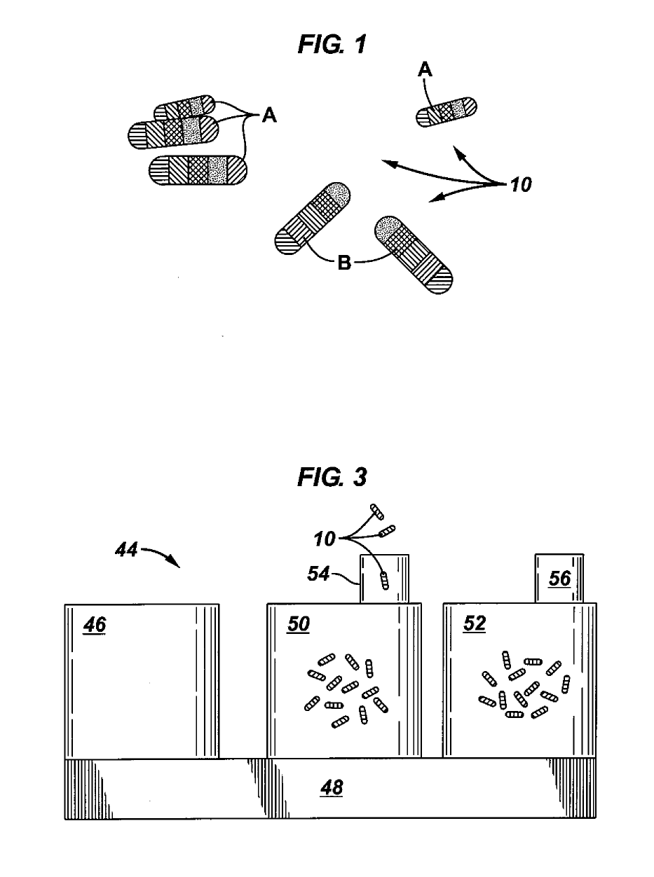

FIG. 1 shows barcoding written onto micrometer size particles 10 according to aspects of the invention. The particles 10 were illuminated with a UV light source and viewed through a 420-nm long-pass filter, as described in the Dejneka Papers. Two sets A, B of particles 10 are clearly revealed. The large number of combinations that can be encoded on the particles, their compatibility with solvents, their miniature size, and their ruggedness makes the RE-doped particles 10 highly suitable for various subsurface applications.

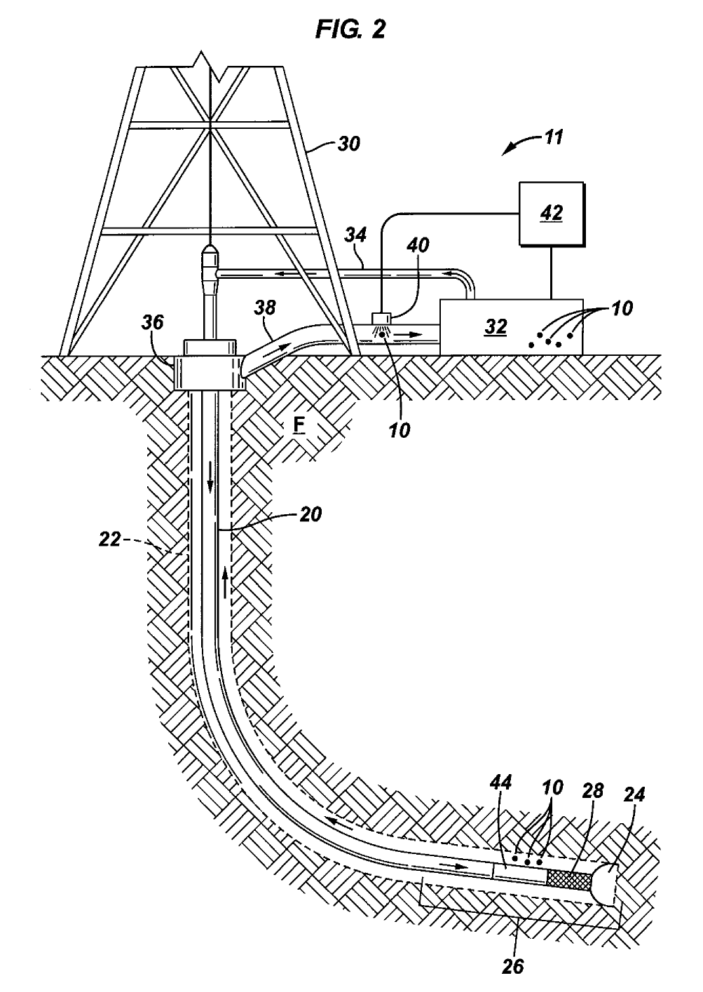

Aspects of the invention comprise the use of the particles 10 to trace fluids and solids in a subsurface environment and to provide means of communication and monitoring. FIG. 2 shows an aspect of the invention. A system 11 includes a drill string 20, shown disposed within a borehole 22 traversing a subsurface formation F as the hole is cut by the action of the drill bit 24 mounted at the far end of a bottom hole assembly (BHA) 26. The BHA 26 is attached to and forms the lower portion of the drill string 20. BHA 26 contains a number of devices including various subassemblies 28 including those used for measurement-while-drilling (MWD) and/or logging-while-drilling (LWD). Information from the subassemblies 28 is communicated to a telemetry assembly (not shown) in the drill string 20 which conveys the information to the surface as is known in the art (e.g., via pressure pulses through the drilling mud).

At the surface, the system 11 includes a derrick 30 and hoisting system, a rotating system, and a mud circulation system. Although this aspect of the invention is shown in FIG. 2 as being on land, those skilled in the art will recognize that the present invention is equally applicable to marine environments. A mud circulation system pumps drilling fluid down the central opening in the drill string 20. The mud is stored in mud pit which is part of a mud separation and storing system 32. The mud is drawn in to mud pumps (not shown) which pump the mud though stand pipe 34 and into the Kelly and through the swivel.

The mud passes through drill string 20 and through drill bit 24. As the drill bit grinds the formation into cuttings, the mud is ejected out of openings or nozzles in the bit with great speed and pressure. These jets of mud lift the cuttings off the bottom of the hole and away from the bit, and up towards the surface in the annular space between drill string 20 and the wall of the borehole 22, as represented by arrows in FIG. 2. At the surface the mud and cuttings leave the well through a side outlet in a blowout preventer 36 and through a mud return line 38. The mud return line 38 feeds the mud into the separation and storing system 32, which separates the mud from the cuttings. From the separator, the mud is returned to a mud pit (not shown) for storage and re-use.

According to aspects of the invention, coded particles 10 are disposed in the mud separation and storing system 32, such that they are set for selective release to a subsurface location via the mud flow. A particle detection unit 40 is coupled into the mud return line 38 and linked to surface equipment 42 comprising computer, display, recording, and user interface means as known in the art. The detection unit 40 includes a radiation source (e.g., UV light source), one or more camera devices, and optics to provide appropriate wavelength illumination to the passing particles 10 in order to resolvable an optical emission such that the individual particle codes are distinguished. An aspect can be implemented wherein the detection unit 40 is incorporated with a filtering or separating device, such as a centrifuge, to collect the particles 10 for analysis. In aspects wherein the particles 10 comprise a ferromagnetic material, the detection unit 40 can be implemented with means to generate a magnetic field (e.g., permanent magnet or electromagnet) to collect the particles for analysis.

Upon resolution of the particle coding, the codes can be matched against a reference database or “code chart.” The detection and identification of the particles 10 can be assisted by the use of a camera that can be used to record images or display on a screen. An aspect of the detection unit 40 may comprise a conventional camera configured to record and display images on a screen. Other aspects may be implemented with a camera disposed on a downhole component to perform subsurface imaging/recording as known in the art. The surface equipment 42 may be configured with a program to process the resolved code data, establish the code matching, track particle travel times, automatically trigger selected particle release, and respectively transmit/receive data/commands to/from remote locations. In aspects comprising particle 10 imaging, the surface equipment 42 may be configured with programming to perform image analysis for particle identification. In yet other aspects, the surface equipment 42 may be configured with programming to calculate the density of particular particles 10. In some aspects, a simplistic system can be implemented wherein the particles 10 are initially disposed in the mud manually and captured in the return line 38 (e.g., using a screening filter, magnet means, centrifuge or separator) for processing by rig personnel. The miniature size and structure of the particles 10 allows them to survive destruction in the drilling process.

In other aspects of the invention, a system may be implemented wherein the particles 10 are set in a release mechanism disposed on the BHA 26, or anywhere along the drill string 20, such that they are selectively or automatically released downhole at a desired depth or when a predetermined event occurs. FIG. 2 illustrates such an aspect of the invention. The BHA 26 may be implemented with a tool comprising a particle release unit 44. Turning to FIG. 3, an aspect of the particle release unit 44 is shown.

An aspect of the particle release unit 44 may be implemented with a sensor 46 adapted to sense a subsurface characteristic or condition (e.g., pressure, temperature, fluid composition, flow rates, etc.). Sensors of these types are well known technology, as are the means to power the sensors. Sensor 46 is in communication with a processor 48 which may comprise a number of microprocessors. One or more chambers 50, 52 contain the particles 10. Associated with the chambers 50, 52 are release mechanisms 54, 56. Under control of processor 48, the release mechanisms 54, 56 can be activated to selectively release the respective particle(s) 10. The release mechanisms 54, 56 may be configured to release the particles) 10 via a forced or pressurized ejection, via direct exposure of the particles to the mud flow, or some combination of these methods as known in the art. In some aspects, the release mechanisms 54, 56 may be instructed to release the particles 10 in accordance with a program in the processor 48. In this manner, the release mechanisms 54, 56 can be instructed to selectively release their particles 10 when different predetermined thresholds or conditions are determined by the sensor 46, or based on input from other sensors in the system.

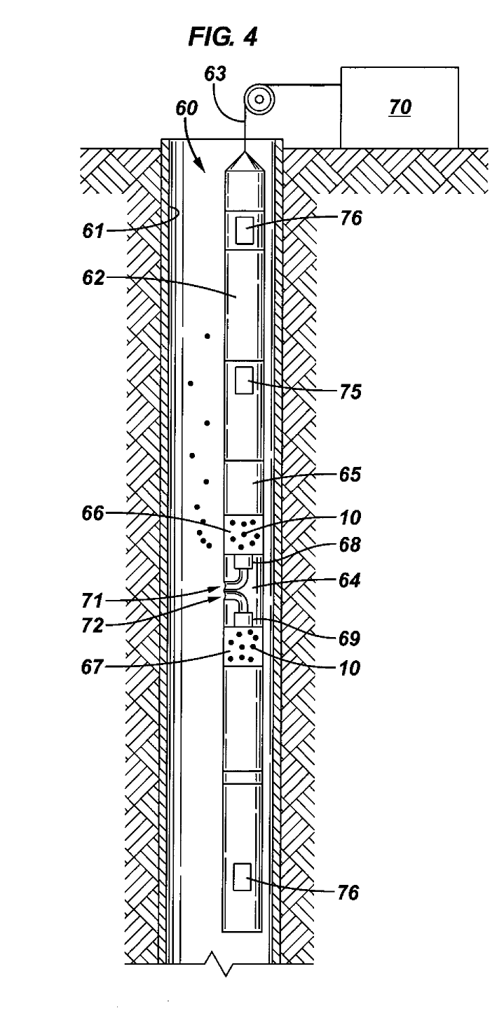

FIG. 4 shows another aspect of the invention. A system 60 of the invention is shown for use within a typical cased production well 61. A downhole tool 62 comprising an elongated body is suspended from a logging cable 63 or wireline which is spooled on a powered winch (not shown) at a surface location adjacent to the well 61. As is usual, the cable 63 is configured with one or more conductors that are cooperatively coupled to surface instrumentation 70 for power/signal communication and recordation as a function of time/depth. The tool 62 includes a particle release unit 64 selectively controllable by way of the surface instrumentation 70 or via signals from a processor 65 in the tool. The particle release unit 64 includes upper and lower enclosed chambers 66, 67 spatially disposed within the tool 62 body to respectively contain the particles 10 under pressure. The chambers 66, 67 are configured for selective and repetitive discharge of particles 10 into the well bore.

To control the release of the particles 10 from their respective chambers 66, 67, the release unit 64 includes valves 68, 69 that are coupled to each of the chambers and respectively arranged, upon being opened, to selectively communicate the chambers with discharge ports or laterally-directed orifices 71, 72. The particles 10 are maintained at elevated pressures which exceed the well bore pressure at the release depth location of the tool 62. As depicted in FIG. 4, an aspect of the tool 62 may also include one or more sources/sensors 75 comprising conventional measurement means as known in the art. It will be appreciated by those skilled in the art that other particle release units may be devised with various types of mechanisms and in different configurations to implement the aspects of the invention disclosed herein. For example, U.S. Pat. No. 6,125,934 and U.S. Patent Publication No. 20070144737 (both assigned to the present assignee and entirely incorporated herein by reference) describe downhole tools equipped for subsurface tracer release, tools which can be readily implemented with particles 10 of the invention as disclosed herein.

Aspects of the invention may also be configured to detect subsurface fluorescence emission of the particles 10. Instruments configured to detect fluorescence downhole are known in the art. U.S. Pat. No. 6,704,109 (assigned to the present assignee and entirely incorporated herein by reference) describes a tool equipped with a probe system to illuminate crude oil in the well and detect the emitted fluorescence. Aspects of the invention can be implemented with similar optical systems such that the particles 10 can released, irradiated, and observed downhole. The optics and light sources in these conventional systems are already configured to provide illumination of appropriate wavelength, or they can be readily adjusted to output the desired radiation. In one aspect, the tool 62 of FIG. 4 can be implemented with downhole fluorescence detector units 76 mounted at longitudinally-spaced intervals above and below the particle release unit 64. Such embodiments can be used to detect the particles 10 downhole and provide the data to the surface instrumentation 70 whenever there is particle movement past a detector 76. Alternatively, a tool (e.g., tool 62) equipped with one or more downhole fluorescence detector units 76 may be used to illuminate and detect particles 10 previously released or affixed to the borehole/casing wall, such as particles 10 disposed in proppant/fracturing compounds and stuck in fissures or mudcake. Another aspect of the tool 62 may include an extendable arm (not shown) configured to press the tool, and the detector units 76, against the borehole or casing wall, as known in the art. Yet another aspect of the tool 62 may be configured with the detector units 76 comprising camera means to image the illuminated particles 10.

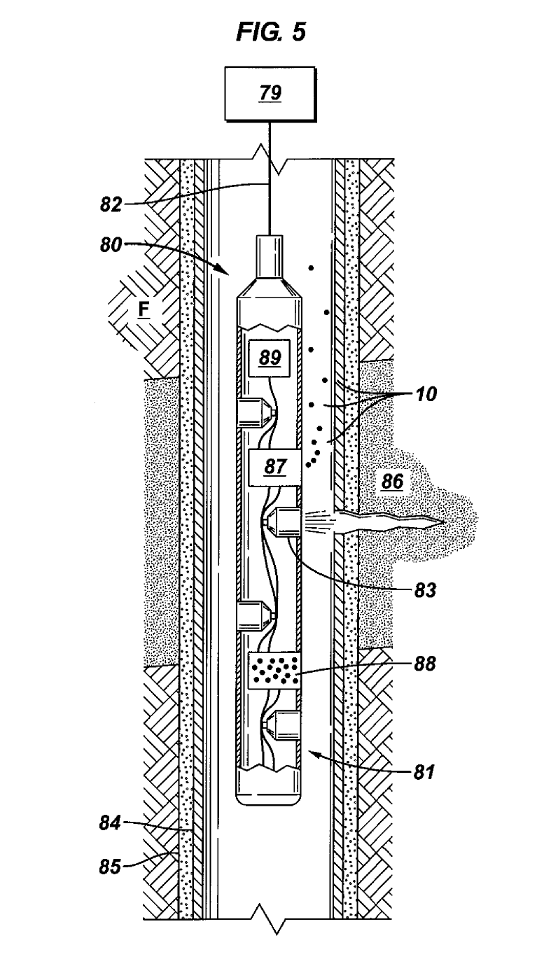

FIG. 5 shows another aspect of the invention. A system 80 includes a perforation tool incorporating releasable particles 10. A perforation gun 81 is suspended from a wireline 82 linked to surface equipment 79 via conventional deployment hardware. The perforation gun 81 comprises essentially a plurality of shaped charges mounted on the gun frame. One of the charges 83 is shown in FIG. 5 firing. The firing charge produces a perforation through the casing 84 and cement 85 into the reservoir region 86 in the subsurface formation F. One or more particle release units 87, 88 are provided to detect the firing of each shaped charge and release the particles 10. In FIG. 5, particle release unit 87 is shown releasing particles 10. Another aspect of the invention may be implemented with the particles 10 incorporated into the charges themselves, such that they are automatically released when the charge is fired (not shown). As with the other systems of the invention, these aspects may be configured for selective release of the particles 10 from the surface and/or via processor means 89 disposed in the gun 81. One use of this system 80 is to provide positive communication to the surface that a charge was properly fired.

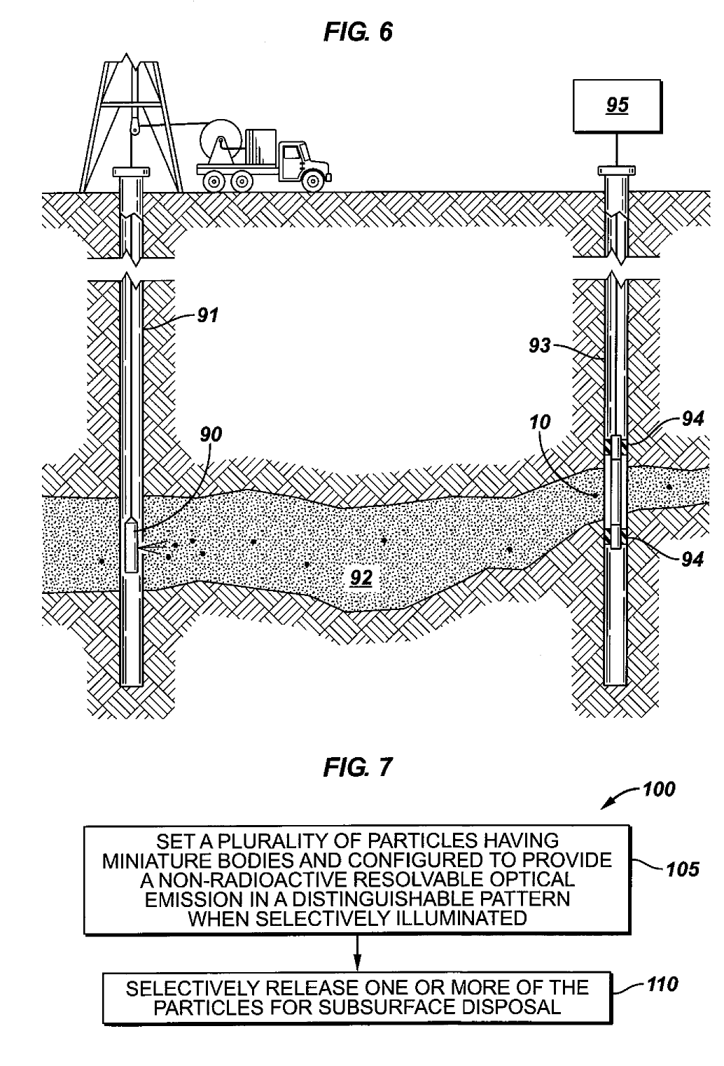

FIG. 6 shows another aspect of the invention. In this aspect the coded particles 10 are used for cross-well applications. A tool 90 containing the particles 10 is disposed in a first well 91 and activated to release the particles at a desired time and depth. As shown in FIG. 6, the first well 91 traverses an oil (or water) zone 92 that extends across a field and is traversed by the path of a second well 93. The second well 93 is shown comprising a pair of conventional packers 94 set in place within the well to restrict inflow to the well within a specific depth range including the zone 92. Surface equipment 95 at the second well 93 is used to monitor and record particles 10 detected at the second well. This data can be correlated to the depths and times of particle 10 release at the first well 91, or in combination with particle release from multiple wells in the field. The wells 91, 93 may be configured with appropriate tubing/liners/casing and production equipment as known in the art. The particle-equipped tool 90 may be any downhole instrument implemented with a particle release mechanism as disclosed herein. This aspect of the invention allows one to perform various operations, including but not limited to, tracking and monitoring specific well production, cross-flow monitoring, completion status/performance checks, and reservoir management.

The disclosed aspects of the invention offer a variety of applications for the coded particles 10. In addition to, and further elaborating on, the previously disclosed applications, uses of the coded particles 10 for subsurface applications include, but are not limited to:

Mud logging—The use of differently coded particles added to the drilling mud at different times provides different types of information:
Circulation time at specific time slots. The travel time of different particles can be logged. The time between the release and the detection of the particles can be measured, as well as the travel time between two or more established locations.
Mud loss detection. A dip of the concentration of a given tagged particle in the mud to indicate greater loss of drilling fluid at a particular depth.
Kick location. A surge of the concentration of a given tagged particle in the mud to indicate that that zone is starting to produce.
Mud cake formation estimation.

Mud cake tagging—The use of differently coded particles added to the drilling mud at different times can tag the mud cake as a function of depth that is correlated with the drilling depth. This provides for.
Correlating drilling depth and wireline depth. This may be done by sampling the mud cake at certain depths.
Cement placement identification by analyzing the displaced mud.
Acidizing job/Acid injection monitoring. By analyzing the particles returning from the mud cake one can locate where the treatment is effective.
Perforating monitoring. Produced particles can be analyzed to correlate the position of perforations.
Clean up treatment monitoring. The amount and type of debris may be estimated using tagging with the particles.

Drill bit communication—In cases where mud pulse telemetry cannot be used, a sub near the drill bit (e.g., unit 44 in FIG. 2) can selectively release a combination of coded particles into the mud to convey information from the drill bit to the surface.

Proppant placement monitoring—Different types of coded particles can be added to the proppant in the fracturing fluid at different times. The concentration of the returned or produced particles of each type will give the efficiency of the fracturing operation.

Gravel pack monitoring—Different types of tagged particles can be added to the gravel at different times during the gravel packing operation. The effectiveness of the placement at different stages of the operation can be monitored by analyzing the concentration of the different particle types returned to the surface. This can also be monitored during production and any eventual sand production. This can be used to identify which region of a gravel pack has failed, for example.

Completion operation monitoring—A sub near a given element of the completion (packer, flow control valve, latching mechanism, etc.) could selectively release a combination of tagged particles into the produced fluid to convey information to the surface. This could contain information about the status of the particular device. Well treatment monitoring. Particles can be mixed with solid acids or other compounds to track/monitor completion operations.

Flow measurement (Production Logging Techniques, slick line, permanent)—The release of tagged particles into the flow can be used to obtain flow velocity. In such aspects, the particles' surface can be treated as known in the art to increase their affinity to a given fluid when multi-phase flows are measured.

Field-scale monitoring—Particle release can be used for injection identification/monitoring, acid injection monitoring, water frontfback allocation, diversion detection, multi-zone stimulation.

Gas market measurements—Particles can be used to track fracturing fluids in tight gas shale ore.

General testing—Particles can be sent from the surface or selectively released downhole to test the operation of downhole instruments or to determine/monitor downhole conditions. Particles can be added to the mud, cement, acid, injection fluid, produced fluid, fracturing fluid, proppant, treatment fluid, gravel, etc. The location of an event can be determined by the type and concentration of particles detected. Different particle sizes can be used in combination to perform any of the operations disclosed herein. For example, the use of different sized particles allows for determination of the size of a fracture, fault, porous medium, etc., that serves as a conduit to the fluids or particles.

FIG. 7 shows a flow chart of a tagging method 100 according to the invention. In one aspect, at step 105 a method entails setting (e.g., within a tool, subsurface, or surface location as disclosed herein) a plurality of particles 10, each particle having a miniature body and configured to provide a non-radioactive resolvable optical emission in a distinguishable pattern when selectively illuminated. Illumination may be provided at the surface or subsurface as disclosed herein. At step 110, one or more of the particles 10 is selectively released for subsurface disposal. Selective release of the particle(s) may be triggered via control signals from the surface, from a subsurface processor programmed for automated release, or a combination of both means as disclosed herein. The method can proceed to, and/or entail, any of the disclosed operations/applications using any of the systems/configurations disclosed herein.

It will be apparent to those skilled in the art that aspects of the invention may be implemented using one or more suitable general-purpose computers having appropriate hardware and programmed to perform the techniques disclosed herein. The programming may be accomplished through the use of one or more program storage devices readable by the computer processor and encoding one or more programs of instructions executable by the computer for performing the operations described above. The program storage device may take the form of, e.g., one or more floppy disks; a CD ROM or other optical disk; a magnetic tape; a read-only memory chip (ROM); and other forms of the kind well known in the art or subsequently developed. The program of instructions may be “object code,” i.e., in binary form that is executable more-or-less directly by the computer; in “source code” that requires compilation or interpretation before execution; or in some intermediate form such as partially compiled code. The precise forms of the program storage device and of the encoding of instructions are immaterial here. Thus these processing means may be implemented in the surface equipment, in the system tools, in a location remote from the well site (not shown), or shared by these means as known in the art. Aspects of the invention may also be implemented using conventional display means situated as desired to display the processed or raw data/images as known in the art.

While the present disclosure describes specific aspects of the invention, numerous modifications and variations will become apparent to those skilled in the art after studying the disclosure, including use of equivalent functional and/or structural substitutes for elements described herein. For example, it will be appreciated that the tools and systems comprising the disclosed aspects of the invention may be implemented for use with any means of subsurface conveyance and in any subsurface operation (e.g., via slick line, coiled tubing, while-tripping, while casing, logging while casing, in conjunction with wired drill pipe, etc.). All such similar variations apparent to those skilled in the art are deemed to be within the scope of the invention as defined by the appended claims.

What is claimed is:

1. A tagging system, comprising:
a plurality of particles, each particle having a miniature body;
each particle configured to provide a non-radioactive resolvable optical emission in a distinguishable pattern when selectively illuminated; and
wherein one or more of the particles is set for selective release to a subsurface location.

2. The system of claim 1, wherein each particle is configured to provide a coded pattern of fluorescence emission in response to illumination by a light source.

3. The system of claim 2, wherein one or more of the particles is set for selective release from a surface location for transit to a subsurface location.

4. The system of claim 2, wherein one or more of the particles is set for selective release from a tool disposed subsurface.

5. The system of claim 2, wherein at least one particle has a body comprising silicon oxide, germanium oxide, aluminum oxide, boron oxide, glass, crystal, or a ferromagnetic material.

6. The system of claim 1, further comprising a light source disposed at the surface or configured for subsurface disposal.

7. The system of claim 1, further comprising a unit to collect at least one of the particles, wherein the unit comprises a magnet, a filter, a separator, or a centrifuge.

8. The system of claim 1, further comprising a camera to image at least one of the particles.

9. The system of claim 1, further comprising a processor configured to analyze image data of a particle, identify a particle, or calculate a density of a particle.

10. A tagging method, comprising:
setting a plurality of particles, each particle having a miniature body and configured to provide a non-radioactive resolvable optical emission in a distinguishable pattern when selectively illuminated; and
selectively releasing one or more of the particles for subsurface disposal.

11. The method of claim 10, wherein each particle is configured to provide a coded pattern of fluorescence emission in response to illumination by a light source.

12. The method of claim 11, wherein selectively releasing one or more of the particles comprises releasing from a surface location for transit to a subsurface location.

13. The method of claim 11, wherein selectively releasing one or more of the particles comprises releasing from a tool disposed subsurface.

14. The method of claim 11, wherein at least one particle has a body comprising silicon oxide, germanium oxide, aluminum oxide, boron oxide, glass, crystal, or a ferromagnetic material.

15. The method of claim 10, further comprising illuminating at least one of the particles with a light source disposed at the surface or at a subsurface location.

16. The method of claim 10, further comprising imaging at least one of the particles.

17. The method of claim 10, further comprising releasing one or more of the particles in a first borehole traversing a subsurface formation and observing for a released particle at a second borehole location.

18. The method of claim 10, further comprising using at least one of the particles to determine a fluid flow property, determine a perforation, determine a kick location, determine mud cake formation, determine an event location, determine a tool status, determine a depth location, determine a transit time, monitor cement placement, monitor fracturing efficiency, monitor proppant placement, monitor injection, monitor well treatment, monitor gravel packing, or convey information.

19. The method of claim 10, further comprising adding at least one of the particles to a fluid, fluid mixture, cement, acid, proppant, or gravel.

20. The method of claim 10, further comprising determining the time between the release and the detection of at least one of the particles.

21. An apparatus, comprising:
an elongated body configured for subsurface disposal; and
the body having at least one chamber to house a plurality of particles therein, each particle having a miniature body and configured to provide a non-radioactive resolvable optical emission in a distinguishable pattern when selectively illuminated.

22. The apparatus of claim 21, wherein each particle is configured to provide a coded pattern of fluorescence emission in response to illumination by a light source.

23. The apparatus of claim 21, wherein the body is configured for subsurface disposal on cable means or linked to a drill collar.

24. The apparatus of claim 21, the body further comprising a particle release unit, a light source, a camera, or a fluorescence detector.

25. The apparatus of claim 21, wherein at least one particle has a body comprising silicon oxide, germanium oxide, aluminum oxide, boron oxide, glass, crystal, or a ferromagnetic material.
